(19) United States
(12) Patent Application Publication
(10) Pub. No.: US 2008/0124312 A1
(43) Pub. Date: May 29, 2008

(54) Polyamine Compositions

(76) Inventor: Erik Lovaas, Oslo (NO)

Correspondence Address: NEEDLE & ROSENBERG, P.C., SUITE 1000, 999 PEACHTREE STREET, ATLANTA, GA, 30309-3915, US

(21) Appl. No.: 11/666,937

(22) PCT Filed: Nov. 7, 2005
(86) PCT No.: PCT/GB05/04279

§ 371 Date: Sep. 24, 2007

(30) Foreign Application Priority Data
Nov. 5, 2004 (NO) 20044818

(51) Int. Cl. A61K 38/43; A61K 31/132; A61Q 19/08

(52) U.S. Cl. 424/94.1; 514/674

(57) ABSTRACT

The present invention provides a method of inhibiting formation of solid hydrates in a conduit used to contain a flowing, water-containing hydrocarbon, which method comprises introducing into said hydrocarbon in said conduit a gas which raises the pressure threshold for hydrate formation for said water-containing hydrocarbon, said gas being introduced in a manner which causes it to travel along at least part of said conduit in the direction of hydrocarbon flow.

This invention relates to topical cosmetic or cosmeceutical compositions containing polyamines, to their use in methods of cosmetic or cosmeceutical treatment and to the use of polyamines for their manufacture.

Skin is a highly metabolic tissue, which possesses the largest surface area in the body and serves as the protective layer for internal organs. It is designed to give both physical and biochemical protection and is equipped with a large number of defense mechanisms. Skin is rich in lipids, proteins and DNA, all of which are components which may be degraded.

In very general terms, skin is composed of cells submerged in an extracellular matrix composed of fibrillar components such as collagens and elastin, and nonfibrillar gel components such as glycosaminoglycans (implicated in skin tones and hydration). The extracellular matrix is synthesized in a specific type of cells called fibroblasts, which reside in the matrix. Collagens and elastin form a 3-dimensional network constitutive of the architectural basis of the dermis, in which are dispersed the other susbtances and cells. Fibrillar collagens are the most abundant macromolecules of connective tissues. Their main functions are to ensure the mechanical properties and the structural integrity of the tissue. Elastin is characterized by its high physical and chemical strength and is especially involved in skin suppleness and plasticity. Elastin may be particularly sensitive to ageing: the microfibrillar scaffold of the elastin network is composed of fibrillin, which is a large glycoprotein with a multidomain structure. When elastin is stretched, the immediate tendency is to return to the initial position, with an elastic behaviour. This elasticity decreases with time for different reasons. Natural ageing of the skin is one cause, but several factors exist that accelerate or modify the natural process, such as sun exposure (“extrinsic ageing”).

The most visible signs of ageing of skin is loss of elasticity and loss of extracellular matrix. At the molecular level, ageing is accompanied by an ever increasing formation of intermolecular cross-links in collagen and elastin. These cross-links are useful in youth to provide an optimum function. As time goes by however, the controlled cross-linking process is overtaken by uncontrolled events leading to a loss of contractile properties, elasticity, tone and skin firmness. The result is skin wrinkling and a rough skin surface.

Ageing starts at a young age, but the underlying structural changes can only be detected histologically prior to middle age. Clinically visible changes become evident between about 35 to 45 years of age, and become more and more pronounced thereafter.

Age pigments, which accumulate with chronological age in the nervous system, muscle and skin, represent one of the most striking subcellular modifications in ageing animals. No specific lesion has yet been associated with their presence; neither has any positive attributes of their presence been described. Age pigments, also termed lipofuscin, ceroid, wear and tear pigment, chromolipid, etc., are identifiable by their characteristic fluorescence, they are located inside cells as a yellowish brown, membrane-rich heterogeneous material, and they have characteristic dimensions of 1-5 micrometers.

Such age-related skin pigmentations, often referred to as liver spots, are one of the distressing aspects of ageing and there is a particular need for a preventative or curative treatment for this.

Efforts to prevent ageing, and in particular ageing of the skin, are probably as old as humanity itself. Over the millenia, numerous remedies have been suggested, some of them rather bizarre.

Many people are distressed by or wish to avoid the occurrence of signs of ageing noticeable in the skin and as a result there is a great demand for topical skin treatment products which combat, i.e. prevent, delay, lessen, reduce or eliminate skin ageing effects. By way of a simple example, creams containing alpha-hydroxy acids have in recent decades been available which irritate the skin and so cause a plumping effect and reduce the appearance of skin wrinkles.

Polyamines, i.e. polyazaalkanes, have long been known to exert an antioxidant effect and have been proposed as components for topical skin treatment products. One example of such a polyamine is spermine (1,5,10,14-tetraazatetradecane), a compound that occurs naturally in mammalian semen (see EP-A-209509). The use of such polyamines in skin treatment products is known for example from EP-A-884046 which proposes a photoprotective skin treatment composition (e.g. a sun protection balm) containing a small percentage of spermine.

There remains however a need for further skin treatment compositions capable of combating other effects associated with skin ageing and we have now realised that spermine and other polyamines may be used to achieve quite unexpected skin care effects.

In particular, topically applied spermine may achieve effects such as: reducing, delaying or preventing development of age-related skin pigmentation (e.g. production of lipofuscin and hence development of “liver spots”); reducing, delaying or preventing glycosaminoglycan degradation and hence maintaining or enhancing skin smoothness; improving epidermal capilliary blood flow (and hence improving skin colour); and reducing, delaying and preventing degradation of skin elasticity.

In particular:
1. Polyamines prevent damage to the elastic fibres of skin, thus preserving skin elasticity;
2. Polyamines slow down the formation of age pigments;
3. Polyamines protect hyaluronic acid against degeneration, and preserve the water-binding capacity of the epidermis; and
4. Polyamines stimulate blood flow in the outer capillaries of the epidermis, improving the flush of the skin and at the same time stimulating the metabolic processes in the epidermis.

Thus viewed from one aspect the invention provides the use of an unbranched aliphatic polyamine for the manufacture of a topical skin treatment composition for use in the topical treatment of skin to achieve at least one of the effects of combating age-related skin pigmentation, promoting skin elasticity, enhancing skin colour, and enhancing skin smoothness.

Viewed from a further aspect the invention provides a method of cosmetic treatment of a subject to achieve at least one of the effects of combating age-related skin pigmentation, promoting skin elasticity, enhancing skin colour, and enhancing skin smoothness, which method comprises topically applying to the skin of said subject an effective amount of an unbranched aliphatic polyamine.

The polyamine used according to the invention is clearly not used in the form of a naturally occurring bodily fluid, e.g. semen, but will generally be an isolated pure substance, formulated in a sterile composition with appropriate cosmetic or pharmaceutical carriers or excipients.

The subject treated according to the invention may be any mammal, but humans are intended as the normal subjects, particularly adult humans, more especially aged 35 or more.

In an especially preferred embodiment, the method of the invention is a method of treatment of a subject to combat age-related skin pigmentation, which method comprises topically applying to the skin of said subject, e.g. a subject having visible age-related skin pigmentation, an effective amount of an unbranched aliphatic polyamine.

Viewed from a yet further aspect the invention provides a topical skin treatment composition comprising an unbranched aliphatic polyamine and at least one physiologically tolerable carrier or excipient, together with instructions for the topical application thereof to achieve at least one of the effects of combating age-related skin pigmentation, promoting skin elasticity, enhancing skin colour, and enhancing skin smoothness. Such instructions may typically be provided on the external packaging, as an insert within the external packaging or on the composition container itself.

Viewed from a further aspect the invention provides a topical skin treatment composition comprising at least one physiologically tolerable carrier or excipient, a first unbranched aliphatic polyamine and a further active agent selected from the group consisting of: polyazaalkanes other than said first unbranched aliphatic polyamine, dimethyl sulphoxide, keratolytic agents, unsaturated fatty acids (e.g. omega-3, omega-6 and omega-9 unsaturated fatty acids, especially omega-3 acids, for example EPA, DHA and ALA) and derivatives (particularly esters) thereof, HMG-CoA reductase inhibitors, piperic acid, 8-hexadecene-1,16-dicarboxylic acid, natural triterpenes, Coenzyme Q10 (ubiquinone), vitamin B₃, aloe, acetylglucosamine esters, ACE inhibitors, angiotensin receptor antagonists, eugenyl glycosides, Mallotus japonicus extract, hydroxyacids (e.g. alpha hydroxy acids such as glycolic acid), beta-(1,3) glucans, frog extract, extract of unpolished rice, urea, pine seed oil, marine collagens, plant cell extracts, ursolate and eugenol derivatives, ceramides, cholesterol, glutathione, carnitine, oxygen scavangers, phytosphingosine, calcium channel inhibitors, sucrose linolenate, caffeine, catalase, Rosa mosqueta oil, glycine, Shea butter, perfluoro polyethers, cystein derivatives, and acetylated hyaluronic acid and alpha-amino acids, and salts of any of these.

Particularly preferred active ingredients besides the polyazaalkanes include Coenzyme Q10, Vitamin B3, alpha-hydroxy acids, unsaturated fatty acids (e.g. omega-3, omega-6 and omega-9 unsaturated fatty acids, especially omega-3 acids, for example EPA, DHA and ALA) and derivatives (particularly esters) thereof, catalase, and Rosa mosqueta oil.

In particular the invention provides a topical composition containing: two or more unbranched aliphatic polyamines; or an unbranched aliphatic polyamine and catalase; or an unbranched aliphatic polyamine and vitamin B3; or an unbranched aliphatic polyamine and Rosa mosqueta oil; or an unbranched aliphatic polyamine and coenzyme Q10; or an unbranched aliphatic polyamine and an unsaturated fatty acid (e.g. an omega-3, omega-6 and omega-9 unsaturated fatty acid, especially an omega-3, for example EPA, DHA and ALA) or a derivative (particularly an ester) thereof; or an unbranched aliphatic polyamine and an alpha hydroxy acid.

The polyamines used according to the present invention are preferably amine group terminated linear structures. Desirably they are unbranched aliphatic compounds which occur naturally. The polyamines preferably have (CH₂)ₙ groups linking the nitrogens where n is 2 to 6, especially 3 or 4, and particularly ones comprising 2 to 6 nitrogens, particularly 2, 3 or 4 nitrogens. These polyamines are available from natural sources, e.g. mammalian semen or fermentation products (for example from soy or anchovies), or may be manufactured by conventional techniques, e.g. solid state polypeptide production followed by amidation and reduction. It is particularly preferred to use naturally occurring polyamines, e.g. putrescine (H₂N(CH₂)₄NH₂), cadaverine (H₂N(CH₂)₅NH₂), spermidine (H₂N(CH₂)₃NH(CH₂)₄NH₂), and spermine (H₂N(CH₂)₃NH(CH)₄ NH(CH₂)₃NH₂), more particularly putrescine, spermidine or spermine, and especially spermine. The use of a combination of two such polyamines, e.g. in a mole ratio of 1:99 to 99:1 especially 10:90 to 90:10, is especially preferred (e.g. spermine and spermidine) as is the use of a combination of three or more such polyamines, for example with each present at 1 to 100% mole relative to the most abundant, especially 10 to 100% mole, particularly 30 to 100% mole.

The use of dibutylenetriamine, tributyltetramine, 1,6,10,15-tetraazapentadecane, 1,5,9,13-tetraazatridecane and 6-aminobutyl-1,6,11-triazaundecane may also be considered.

In the polyamines used according to the invention the average carbon chain length, i.e. the carbon chain between heteroatoms, may be as low as 1 or 2; however, where this average is below 3.0 the polyamine is preferably a minor component of the composition, e.g. no more than 5% wt, preferably no more than 1% wt.

In general, in the polyamines of the invention the average carbon chain length is preferably at least 2.5, more preferably at least 3.0, especially at least 3.25, e.g. 3.25 to 6.0.

Desirably the polyamines used according to the invention have molecular weights in the range 88 to 202 Da.

The polyamine used in accordance with the invention may conveniently be in salt form with a physiologically tolerable counterion, e.g. an organic acid, particularly preferably an alpha-hydroxyacid or fatty acid. Such salts, which may be prepared by reaction of the polyamine and the acid, e.g. in solution for example in approximately equimolar amounts, are novel and form a further aspect of the invention as do topical compositions containing them and a carrier or excipient.

In the compositions of or used according to the invention, the total polyamine content is preferably 0.0005 to 5% wt, more preferably 0.001 to 1% wt, especially 0.005 to 0.5% wt, particularly 0.01 to 0.08% wt, more particularly 0.02 to 0.06% wt, especially 0.03 to 0.05% wt, e.g. 0.04% wt (i.e. 400 ppm).

The compositions of the invention preferably do not contain multivalent metal (e.g. transition metal) ions in otherwise labile form at concentrations of above 10% mole relative to the polyamine, especially 1% mole.

The compositions of or used according to the invention may be in any form suitable for topical application, e.g. creams, gels, solutions, emulsions, dispersions, suspensions etc. and may if desired include a carrier substrate, e.g. a woven or non-woven web. The compositions may contain conventional topical composition components, such as for example, solvents, oils (e.g. plant oils), aromas, colorants, pH modifiers, viscosity modifiers, binders, diluents, emollients, antioxidants, skin irritants, thickeners, vitamins, preservatives, stabilizers, humidifiers, skin penetration enhancers, vesicle wall formers, etc. Examples of suitable formulations include body milks, body lotions, hand creams, sun lotions, and oils.

In one preferred form, the composition used according to the invention is an eyeliner or other eye makeup containing inorganic colorants, e.g. metal oxides, for example transition metal oxides such as iron or chromium oxides. The polyamine may bind to these and thus be present in a sustained release form.

The components of the compositions of the invention will typically be present in conventional concentrations for skin treatment compositions. Active components, i.e. those having a skin protective effect beyond simple moisturization or oiling, will generally be present at concentrations of 0.001 to 20% wt, especially 0.01 to 10% wt, particularly 0.05 to 5% wt.

In a further aspect the invention also provides an aqueous topical skin treatment cream comprising: an unbranched aliphatic polyamine (e.g. spermine) and coenzyme Q10.

The compositions of and used according to the invention are preferably for application to: (a) the hands (especially for combating age-related pigmentation); (b) the breasts; (c) the thin skin under the eyes; (d) the upper arm (especially the surface adjacent the torso); (e) the undersurface of the chin; and (f) the decolletage (i.e. the area exposed by an open-necked shirt). Compositions and methods specific for these regions form further aspects of the invention.

The compositions of and used according to the invention are particularly preferably creams, emulsions, gels, vesicle dispersions, or vesicle forming compositions. In terms of vesicles, liposomes are of particular interest as they can facilitate skin penetration of the polyamine. Liposome formulations may be prepared conventionally, e.g. using commercially available precursors. Equally, the inclusion of keratolytics and skin penetration enhancers, e.g. DMSO, is of particular interest, as is the inclusion of vitamins such as vitamin A, vitamin C, vitamin B₆ and vitamin E and derivatives thereof.

As the polyamines may be electrically charged, e.g. by the inclusion of quaternary amine functions or by protonation of amine nitrogens, the compositions may deliver the polyamine transdermally under the action of an electric field, i.e. by iontophoresis. The compositions may thus conveniently be presented in gel form within patches provided with electrodes and a battery. This format is of particular interest when the skin treatment desired is localized, e.g. in the treatment of localized skin blemishes.

In general, the compositions should be applied to the skin either prophylactically, i.e. to inhibit development of a skin blemish such as pigmentation or the like, or to the affected skin of a subject in which the skin blemish is already present. In the case of skin pigmentation blemishes, the patient will generally be at least 50 years old, more typically at least 55, especially at least 60, particularly at least 65.

The polyamines will typically be administered at a dosage of about 0.01 to 50 g/m², preferably 0.1 to 10 g/m², especially 1 to 5 g/m². Any other active ingredients will typically be used at from 10% to 200%, preferably 50 to 110%, more preferably 80 to 105% of their normal dosages.

The compositions of and used according to the invention may be produced by standard cosmetic or pharmaceutical composition production techniques, e.g. simple admixture optionally followed by sterilization. The compositions are desirably packaged in single dose units or in units suitable for up to 100 applications, e.g. 2 to 10 applications. The use of sachets, spray dispensers, pump dispensers, and wipes is especially preferred.

The present invention thus advantageously provides cosmeceutical compositions, as well as methods for improving skin health and prevention and treatment of wrinkles, age pigments and other skin disorders. The invention provides a cosmeceutical composition that retards skin ageing and preserves skin elasticity, softens and tones. There is also disclosed a formulation that rejuvenates damaged skin. The invention provides a cosmetic formulation for topical treatment of skin to moderate and retard ageing changes in young age before ageing changes first become evident clinically. The treatment is based on the observation that bioactive polyamines (in particular spermine) prevent deterioration of the elastic material of skin and thus preserve a youthful look. However, the effects are also of fundamental importance for the maintenance of healthy and functional body performance (like keeping the elastic tone of blood vessels) as the polyamines also retard the ageing of skin cells and the formation of age pigments (lipofuscin), and protect glycosaminoglycans (like hyaluronic acid) against degradation. By the latter effect the skin will preserve its binding capacity for water and maintain its natural smoothness. The cosmeceutical composition increases the blood flow of epidermal capillaries, improving the flush of the skin and at the same time stimulating the metabolic processes in the epidermis. The sum of these effects is an overall improvement in the appearance and functionality of the skin. The polyamines are actively taken up by keratinocytes, and thus, in contrast to most other cosmetic components, penetrate the skin. The invention thus accomplishes two goals. First, a prophylactic effect in preventing progression and worsening of the damage with the passage of time. Secondly, various abnormalities are corrected and modified to the extent that the structure and function of the skin acquires the characteristics of younger skin.

Thus the compositions of the invention may desirably contain a skin irritant, e.g. an alpha-hydroxy acid, having a further desirable effect on the skin. Moreover the method of the invention may comprise application of a polyamine simultaneously or before or after application of a composition containing a skin irritant.

The invention will now be illustrated further with reference to the following non-limiting Examples:

### EXAMPLE 1

### Topical Cream

| Ingredient | Parts by weight |
|---|---|
| Water | 61-66 |
| Propylene Glycol Dicaprylate/Dicaprate | 6-8 |
| Ethylhexyl Stearate | 3-4 |
| Prunus Armeniaca | 0.5-1.5 |
| Simmondsia Chinensis | 0.4-0.6 |
| C₁₂₋₂₀ Acid PEG-8 Ester | 8-12 |
| Olus | 3-4 |
| Propylene glycol | 2.5-3.5 |
| Glyceryl stearate | 1.5-2.5 |
| Potassium cetyl phosphate | 0.8-1.2 |
| Glycerin | 0.4-0.6 |
| Sodium PCA | 0.1-0.2 |
| Dimethicone | 1.5-2.5 |
| Spermine | 0.03 |
| Ascorbyl Palmitate | 0.005-0.015 |
| Ubiquinone | 0.08-0.12 |
| PEG-7 Glyceryl Cocoate | 0.05-0.1 |
| Alcohol denat. | 0.05-0.1 |
| Tocopheryl Acetate | 0.05-0.1 |
| Panthenol | 0.05-0.1 |
| Retinyl Palmitate | 0.05-0.1 |
| Helianthus Annuus | 0.05-0.1 |
| Tocopherol | 0.05-0.1 |
| Lactic Acid | 0.5-1.5 |
| Sodium Gluconate | 0.05-0.15 |
| Phenoxyethanol | 0.4-0.6 |
| Sodium Benzoate | 0.2-0.3 |

### The components listed above are mixed and emulsified.

### EXAMPLE 2

### Topical Cream

| Ingredients | Parts by weight |
|---|---|
| Water | 80-85 |
| Alcohol Denat. | 5-10 |
| Propylene Glycol | 2-4 |
| Sorbitol | 1-3 |
| Polyquaternium-10 | 1-3 |
| Dicaprylyl Carbonate | 0.5-1.5 |
| Sodium Hyaluronate | 0.5-1.0 |
| Tocopherol | 0.05-0.1 |
| Tocopheryl Acetate | 0.05-0.1 |
| Spermine | 0.02-0.04 |
| Ubiquinone | 0.008-0.012 |
| Retinyl Palmitate | 0.05-0.1 |
| PEG/PPG-14/4 Dimethicone | 0.3-0.7 |
| Sodium Gluconate | 0.5-1.5 |
| Menthyl Lactate | 0.05-0.15 |
| Phenoxyethanol | 0.3-0.7 |
| Lactic Acid | 0.5-1.5 |
| Propylene Glycol Dicaprylate/Dicaprate | 0.008-0.012 |
| Prunus Armeniaca | 0.008-0.012 |
| Panthenol | 0.05-0.1 |
| Helianthus Annuus | 0.05-0.1 |
| PEG-7 Glyceryl Cocoate | 0.05-0.1 |

### The components are mixed and emulsified.

Further creams are prepared analogously using the weight content mid-points for these ingredients and further including in parts by weight: (A) 0.03 spermidine; (B) 0.07 vitamin B₃; (C) 0.07 catalase; (D) 0.07 Rosa mosqueta oil; (E) 0.03 spermidine, 0.07 vitamin B₃; 0.07 catalase and 0.07 Rosa mosqueta oil.

The six compositions of this example may be applied liberally to the skin area to be treated, e.g. hands, upper arms, neck and chin, and under-eyes, once a day, preferably twice a day.

### EXAMPLE 3

In vivo studies

The efficacy of spermine was evaluated in vivo in a cosmetic formulation containing 400 ppm spermine, first by measuring its effect on the mechanical properties of the skin (elasticity) and then by carrying out an analysis of images, the technique used for observing its effect on the cutaneous surface and, specifically, on wrinkles, before and after treatment.

The cutaneous elasticity of the skin was analyzed by measuring its recovery after applying suction with an SEM 474 Cutometer (Courage & Khazaka). In this study, a constant suction pressure of 350 mbar was used, and measurements were recorded three times, to give elasticity curves showing the parameters R0, R1 and R9. RO is the height of the curve when the suction pressure is applied and RI is the width of the same curve and represents the ability of the skin to return to its initial state after being submitted to the pressure. R9 is cutaneous elasticity, an experimental value obtained from RO and RI.

The test was carried out on a group of six women between 45 and 55 years of age (mean, 50 years) on the area surrounding the eye. The aforementioned cosmetic formulation was applied twice daily for a period of 45 days.

Image analysis is an essential tool for studying skin microtopography. The basic principle consists of measuring shadows generated on the surface of silicona prints by incident lighting. An impression of the skin's surface geometry is obtained by applying a thin layer of silicone to the skin's surface. The rubber impression is lifted from the skin and placed on a level surface with the side containing the skin's imprint facing downwards. The skin replica is placed under a cine camera connected to a personal computer and lighted laterally (26°). Under these experimental conditions, the different grey levels corresponding to the furrow shadows can be recorded and analyzed. By using an image processor with a densitometric measuring program based on 286 grey degree, the corresponding relief was quantified in terms of the mean number of lines and the mean depth of lines.

The duplicate was illuminated laterally and filmed by the cine camera. Its image was transmitted to the processor, which is able to identify the wrinkles (related as negative on the duplicate) and to measure their depth by the color difference and intensity created by the shadows. With such images it is possible to study the cutaneous relief of an area and observe its development under specific treatment. In this study the depth of the wrinkles was examined before and after treatment, and the effect of the cosmetic formulation was compared constantly with a control sample.

### EXAMPLE 4

### Cell Renewal and Elastin Synthesis

the investigation on spermine-stimulated call renewal and elastin synthesis in fibroblasts we used the dermal equivalent (DE) model of Frei et al. (see Int. J. Cosmetic Science 20:159-173 (1998)), as follows: Fibroblasts from human foreskin explants were subcultured in fibroblast culture medium (FCM) consisting of Dulbecco's Modified Eagle's Medium (DMEM) supplemented with 10% neonatal calf serum, 25 mg/L gentamycin, 100,000 UI/1 penicillin, 1 mg/L amphotericin B, 50 mg/L sodium ascorbate and 4 mM L-glutamine (Sigma, St. Louis, USA). Fibroblasts (200,000) from the 4th to the 10th passage were seeded onto each dermal matrix previously rehydrated with FCM and placed in 24 multiwell dishes. Two mL of FCM were added to each well. DE were then incubated at 37° C., in an atmosphere of CO₂/air (5%/95%, v/v) for 3 weeks and the medium was changed twice a week. By the end of this period, the cells had proliferated, migrated and synthesized their own extracellular matrix filling the pores of the dermal substrate.

Spermine was tested for its ability to stimulate fibroblast renewal in the DE model model. DEs were cultured for 2 weeks in DMEM culture medium supplemented by 2% of calf serum and 1.25% (v/v) of peptide (n=6). Control DEs without peptide were tested under the same conditions (n=6).

The cell viability was evaluated by a colorimetric method using MTT after 1 and 2 weeks of culture with the peptide. Viable cells convert the colorless tetrazolium salt MTT to blue formazan that can be measured spectrophotometrically at 570 nm after extraction with dimethylsulfoxide (DMSO). Optical density has been shown to be proportional to the number of viable cells.

The stimulating effect of spermine on the formation of extracellular components (elastin) was investigated on DEs prepared during a period of 20 days. At this time, fibroblasts reached confluency in the dermal substrate and were quiescent. On the 21st day, 400 ppm of spermine was added for 8 days to the DE culture medium in which the concentration of calf serum was decreased to 5% (v/v). Control DEs without spermine were tested in the same conditions. At the end of the experiment, the cell density in the treated and control DEs was evaluated by the MTT method. This test was performed to confirm that spermine had no further effect on cell renewal after the growing phase.

The soluble fraction of elastin was assayed in the culture medium sampled on day 29 (i.e. after the peptide had been present for 8 days), using a specific calorimetric method described by Winkelman and Spicer (see Stain Technol. 37:303-305 (1962)). After precipitation of the soluble tropoelastin present in the culture medium (pool of six DEs control or treated), the precipitate is mixed with a synthetic porphrine. The elastin-dye complex was separated by centrifugation, solubilized, and the optical density measured at 513 nm (n=5) (Fastin, Realef, France).

1-3. (canceled)

4. A method of treatment of a subject to achieve at least one of the effects of combating age-related skin pigmentation, promoting skin elasticity, enhancing skin colour and enhancing skin smoothness, which method comprises topically applying to the skin of said subject an effective amount of an unbranched aliphatic polyazamine.

5. A topical skin treatment composition comprising an unbranched polyamine and at least one physiologically tolerable carrier or excipient, together with instructions for the topical application thereof to achieve at least one of the effects of combating age-related skin pigmentation, promoting skin elasticity, enhancing skin colour and enhancing skin smoothness.

6. A composition as claimed in claim 5 containing a polyamine selected from putrescine, spermidine and spermine.

7. A composition as claimed in claim 5 further containing a further active agent selected from the group consisting of: polyazaalkanes other than said first unbranched aliphatic polyamine, dimethyl sulphoxide, keratolytic agents, unsaturated fatty acids and derivatives thereof, HMG-CoA reductase inhibitors, piperic acid, 8-hexadecene-1,16-dicarboxylic acid, natural triterpenes, Coenzyme QlO (ubiquinone), vitamin B₃, aloe, acetylglucosamine esters, ACE inhibitors, angiotensin receptor antagonists, eugenyl glycosides, Mallotus japonicus extract, hydroxyacids, frog extract, extract of unpolished rice, urea, pine seed oil, marine collagens, plant cell extracts, ursolate and eugenol derivatives, ceramides, cholesterol, glutathione, carnitine, oxygen scavangers, phytosphingosine, calcium channel inhibitors, sucrose linolenate, caffeine, catalase, Rosa mosqueta oil, glycine, Shea butter, perfluoro polyethers, cystein derivatives, and acetylated hyaluronic acid and alpha-amino acids, and salts of any of these.

8. A topical skin treatment composition comprising at least one physiologically tolerable carrier or excipient, a first unbranched aliphatic polyamine and a further active agent selected from the group consisting of Coenzyme QlO, Vitamin B3, alpha-hydroxy acids, unsaturated fatty acids and derivatives thereof, catalase, and Rosa mosqueta oil.

9. A composition as claimed in claim 5 containing: two or more unbranched aliphatic polyamines; or an unbranched aliphatic polyamine and catalase; or an unbranched aliphatic polyamine and vitamin B₃; or an unbranched aliphatic polyamine and Rosa mosqueta oil; or an unbranched aliphatic polyamine and coenzyme QlO; or an unbranched aliphatic polyamine and an unsaturated fatty acid or a derivative thereof; or an unbranched aliphatic polyamine and an alpha hydroxy acid.

10. An aqueous topical skin treatment cream comprising an unbranched aliphatic polyamine and coenzyme QlO.

11. A method comprising applying a topical skin treatment composition comprising an unbranched aliphatic polyamine to the skin of a subject to achieve at least one of the effects of combating age-related skin pigmentation, promoting skin elasticity, enhancing skin color and/or enhancing skin smoothness.

12. The method of claim 11, wherein the unbranched aliphatic polyamine comprises at least one of putrescine, spermidine, spermine, or a combination thereof.

13. The method of claim 11, wherein the topical skin treatment is suitable for use in the topical treatment of skin to combat age-related skin pigmentation.
